(19) United States
(12) Patent Application Publication
(10) Pub. No.: US 2026/0183025 A1
(43) Pub. Date: Jul. 2, 2026

(54) DEVICE FOR TREATING A FRACTURE

(71) Applicant: I.T.S. GMBH, Lassnitzhöhe (AT)

(72) Inventors: Ronald PRAGER, Bovenau (DE); Volker BÜHREN, Murnau (DE)

(21) Appl. No.: 18/857,863

(22) PCT Filed: Nov. 7, 2022
(86) PCT No.: PCT/AT2022/060379

§ 371 Date: Oct. 18, 2024

(51) Int. Cl. A61B 17/74; A61B 17/00; A61B 17/56; A61B 17/86; A61B 17/88; A61B 90/00

(52) U.S. Cl. CPC A61B 17/744; A61B 17/864; A61B 17/8816; A61B 90/03; A61B 2017/00477; A61B 2017/564; A61B 2090/035

(57) ABSTRACT

The present disclosure relates to an apparatus for treating a fracture, comprising a nail which can be introduced into an intramedullary canal of a bone, wherein the nail comprises a bore that is aligned roughly transversely to a longitudinal axis of the nail, wherein a coupling device, is provided which can be introduced into the bore such that the coupling device protrudes past the bore on both sides and can be moved within the bore along a transverse axis in a lateral direction and a medial direction oriented in an opposing manner to the lateral direction, and which coupling device can be fixed in place at an end in a bone part, wherein an adjusting device is provided which can be fixed in place in different positions relative to the nail and wherein the adjusting device comprises a first stop surface that limits a mobility of the coupling device in a lateral direction when the coupling device and adjusting device are arranged on the nail, so that by changing the position of the adjusting device relative to the nail, a lateral end position up to which the coupling device can be moved relative to the nail in a lateral direction can be altered.

The invention relates to an apparatus for treating a fracture, in particular a fracture of a proximal femur such as a fracture of the femoral neck, for example, comprising a nail which can be introduced into an intramedullary canal of a bone, in particular into an intramedullary canal of a femoral shaft, wherein the nail comprises a bore that is aligned roughly transversely to a longitudinal axis of the nail, wherein a coupling device, in particular a bone screw, is provided which can be introduced into the bore such that the coupling device protrudes past the bore on both sides and can be moved within the bore along a transverse axis in a lateral direction and a medial direction oriented in an opposing manner to the lateral direction, and which coupling device can be fixed in place at an end in a bone part, in particular a femoral head, in particular by means of a thread provided on the coupling device.

The invention furthermore relates to a method for treating a fracture of the femoral neck, wherein a nail is introduced into an intramedullary canal of a femoral shaft, after which a coupling device is introduced into the nail, which coupling device is fixed in place in a femoral head.

From the prior art, apparatuses and methods of the type named at the outset have become known in order to treat a fracture of the femoral neck in particular.

Critical to a rapid healing is thereby a certain mobility between the femoral head and the femoral shaft, for which reason an apparatus has become known from the document U.S. Pat. No. 8,172,841B2, for example, which apparatus allows a certain mobility of the coupling device relative to the nail. According to said document, this mobility is achieved by means of a grub screw arranged distally on the nail, which screw slides in a groove of the coupling device.

It has been shown, however, that, with an apparatus of this type, it is not possible to achieve an optimal healing in all patients.

This is addressed by the invention. The object of the invention is to specify an apparatus of the type named at the outset which can be better adapted to the needs of individual patients. Additionally, a method of this type shall be specified.

According to the invention, the first object is attained with an apparatus of the type named at the outset in which an adjusting device is provided which can be fixed in place in different positions relative to the nail, in particular by means of a thread in the adjusting device and in the nail, and wherein the adjusting device comprises a first stop surface that limits a mobility of the coupling device in a lateral direction when the coupling device and adjusting device are arranged on the nail, so that by changing the position of the adjusting device relative to the nail, a lateral end position up to which the coupling device can be moved relative to the nail in a lateral direction can be altered.

In the course of the invention, it was found that the same mobility of the coupling device relative to the nail, or a play in a transverse direction between the coupling device and nail, is not equally beneficial for all patients, but rather that different magnitudes by which the coupling device can be moved relative to the nail can be advantageous depending on patient-specific parameters, in particular such as a height, a weight, a bone density, a type of fracture, and the like. According to the invention, this adaptability of the mobility or play, which adaptability promotes healing, is achieved in a constructionally particularly simple manner by an adjusting device that can be fixed in place in different positions relative to the nail, which adjusting device limits a mobility of the coupling device in a lateral direction in a form fit via a first stop surface.

The coupling device can typically be connected to the femoral head by means of a thread provided at an end, and is for this purpose normally embodied as a bone screw. However, the coupling device can also be embodied in any other suitable manner in order to be capable of being connected to a bone part such as the femoral head, for example.

In addition, it shall be understood that the apparatus according to the invention can also be used to treat factures other than a fracture of the femoral neck, in particular also for treating a fracture in the region of the proximal femur.

The transverse axis is typically roughly transverse to a longitudinal axis of the nail, normally at an angle at which an axis of the femoral neck lies to an axis of the femoral shaft, for example 110° to 140°.

The adjusting device can preferably be screwed into the nail, namely preferably distally from the coupling device and, for example, preferably at a slight angle to the transverse axis. Thus, by changing a screw-in depth of the adjusting device, typically embodied as an adjusting screw, a play of the coupling device relative to the nail can easily be altered.

The first stop surface can be formed by only a single surface. However, it can also be provided that the first stop surface is formed by multiple stop surfaces that act in parallel and thus limit a mobility in a particularly effective manner. It has been shown, for example, that, when only a single first stop surface is used, there is a risk that the adjusting device slides along the coupling device and the movement is thus limited less than intended. In particular, it can be provided that the first stop surface is formed by two or three stops acting in parallel, for example a primary, a secondary, and a tertiary first stop. The primary stop can thereby be arranged, for example, at a lateral end of the adjusting device, the secondary stop, for example, roughly centrally on the adjusting device, and the tertiary stop in the region of a medial end of the adjusting device.

It is beneficial if the first stop surface is at least partially formed by a shoulder or a collar, in particular an adjusting screw collar, in a region of a lateral end of the adjusting device. The collar can be embodied as an adjusting screw collar and can be positioned at a lateral end of the adjusting screw, for example in order to bear against the coupling device at a predefined relative position of the coupling device and adjusting device, and to thus prevent a further movement of the coupling device relative to the adjusting device in a lateral direction

Preferably, it is provided that the first stop surface is at least partially formed by a part of a thread of the adjusting device, with which thread the adjusting device can be fixed in place relative to the nail. In particular, a medial end of the thread can serve as a stop for a corresponding surface of the coupling device, in order to limit a movement of the coupling device in a lateral direction.

It has proven effective that the first stop surface is at least partially formed by a part of a thread of the coupling device, with which thread the coupling device can be fixed in place at an end in a bone part. The thread of the coupling device can thus serve as a stop, for example in that a lateral end of the thread bears against the adjusting device in a predefined relative position of the adjusting device and coupling device and thus limits a further movement of the coupling device in a lateral direction.

It is preferably provided that the adjusting device is connected to the nail by a thread, wherein a thread lock, in particular a plastic piece that increases a friction in the thread, is provided in order to prevent a loosening of the adjusting device. An unintended loosening of the adjusting device is thus prevented in a simple manner.

In order to easily prevent errors during an insertion of the apparatus due to excessive screwing-in, it is preferably provided that the adjusting device can only be screwed into the nail up to a predefined position, wherein this is constructively implemented with a wrench in particular, preferably a hex wrench, which, starting at a predefined position of the adjusting device, no longer produces any contact with a corresponding counterpiece, in particular a hexagon socket, in the adjusting device.

It is beneficial if the adjusting device comprises a second stop surface which, when the coupling device and adjusting device are arranged on the nail, limits a mobility of the coupling device in a medial direction.

In addition, it can also be provided that the adjusting device lies at an angle to the coupling device, so that a ramp occurs which defines a mobility of the coupling device via the angle. The mobility of coupling device relative to the nail in a transverse direction can thus be constructively altered via an angle between the adjusting device and coupling device and, while an operation is still in progress, via a position of the adjusting device relative to the nail or a screw-in depth. The angle is 0.8° to 2°, for example.

Alternatively thereto, it could, of course, also be provided that the medial end position of the coupling device relative to the nail is formed by a stop on the nail, which stop prevents a further movement of the coupling device in a medial direction starting at a certain position. For this purpose, a collar or a shoulder can be provided, for example, at a lateral end of the coupling device that projects out of the nail on a lateral side, which collar or shoulder is larger than a diameter of a bore in the nail, through which bore the coupling device is guided through the nail, so that starting at a predefined position, the lateral collar or lateral shoulder bears against the nail, and thus forms a stop which prevents a further movement of the coupling device in a medial direction.

It is preferably provided that a position of the adjusting device relative to the nail can be altered along an adjusting direction, in particular along a straight line. This can be implemented in a simple manner especially if the adjusting device is embodied with an outer thread and the nail with a corresponding inner thread, wherein an axis runs along the adjusting direction.

It is preferably provided that the first stop surface and the second stop surface are aligned at different angles to the adjusting direction so that, when the position of the adjusting device relative to the nail is changed, the lateral end position can be changed by a larger magnitude than the medial end position. Thus, not only can a position of the play of the coupling device relative to the nail be altered by means of a change in the position of the adjusting device relative to the nail as a result, but rather also a size of the play. It can also be provided that through a position of the second stop surface does not change with a position of the adjusting device. This is particularly the case if the second stop surface is embodied by a cylinder envelope surface of an adjusting device embodied as an adjusting screw. In this case, only a size of the play and a position of the first stop surface are altered with a position of the adjusting device or with a screw-in depth of the adjusting screw.

Particularly preferably, it is provided that a position of the adjusting device relative to the nail can be altered along an adjusting direction, wherein the adjusting direction is aligned at an angle of 0.1° to 15°, in particular 0.8° to 2°, to the transverse direction, wherein the adjusting direction and the transverse direction preferably lie in the same plane.

In this manner, a design is achieved in a constructively particularly simple manner, in which design an alteration of the position of the adjusting device relative to the nail has different effects on the position of the first stop surface on the one hand and that of the second stop surface on the other hand. Thus, the first stop surface can, for example, be formed by a surface of the adjusting device roughly perpendicular to the adjusting direction, in particular a thread flank of an adjusting device embodied as an adjusting screw, whereas the second stop surface can be formed by a cylinder surface that runs, for example, rotationally symmetrically with the adjusting direction, in particular a conical surface or a cylinder surface running concentrically with the adjusting direction, or an outer surface of the adjusting device embodied as an adjusting screw. With a correspondingly small angle between the adjusting direction and transverse direction, it thus occurs that a change in the position of the adjusting device in an adjusting direction has a roughly equally large effect on the position of the first stop surface, and almost no effect, or none at all, on a position of the second stop surface.

For example, in the case where the second stop surface is formed by a cylinder surface of the adjusting device that is concentric with the adjusting direction, where the adjusting direction is aligned at an angle to the transverse direction, and where a groove corresponding to the adjusting device is arranged in the coupling device at a corresponding angle, a position of the second stop surface is defined solely by the angle, but, in contrast to the first stop surface, does not change when a screw-in depth of the adjusting device is changed, wherein the first stop surface is formed by a face or a thread flank of the adjusting device, which adjusting device can be embodied as an adjusting screw.

Typically, it is provided that the adjusting device is embodied to be longer than the bore, so that the adjusting device can be connected to the nail in a position protruding past the bore on both sides, wherein the first stop surface is arranged medially from the nail. Typically, the adjusting device runs through the entire nail, for example at an opening of the nail. The opening through which the adjusting device projects through the nail can thereby also coincide with the bore through which the coupling device projects through the nail. The corresponding bore can thus be embodied at a distal region with a thread for the adjusting device.

The coupling device and adjusting device are typically embodied to be roughly elongate. preferably roughly cylindrical, especially since corresponding bores in the femoral head normally also have a cylindrical cross-section.

It is beneficial if the coupling device comprises a groove that interacts with the adjusting device and runs along the coupling device, so that a rotation of the coupling device about the transverse axis is limited, preferably prevented, by the adjusting device when the coupling device and the adjusting device are connected to the nail. This is particularly beneficial since, during a healing of a fracture of the femoral neck, wherein the femoral neck is naturally placed under strain, a torsional load also typically acts on the femoral neck, which load could lead to an undesirable rotation of the coupling device relative to the nail. A rotation of this type about the transverse axis is thus efficiently prevented if a corresponding groove is provided which interacts with the adjusting device such that the adjusting device engages in the groove and therefore prevents a rotation of the coupling device relative to the nail.

In order to achieve a simple construction on the one hand and an especially good contact between the adjusting device and the coupling device on the other hand, it is beneficial if the bore is embodied to receive the coupling device in a proximal region and to receive the adjusting device in a distal region. It shall be understood that the statements distal, proximal, lateral, and medial in this case respectively refer to an intended arrangement of the apparatus on a bone of a human body, typically a femur. If used as intended, the adjusting device is thus arranged below, or distally, from the coupling device.

It is beneficial if the coupling device comprises a through-bore projecting from a lateral end to a medial end, into which through-bore a K-wire can be inserted. The coupling device can thus be introduced into a femoral neck in a particularly precise manner in that initially only one bore with a small diameter is introduced into the femoral neck, after which the K-wire is introduced into said bore with a small diameter, whereupon the K-wire is introduced as a guide for a bore with a larger diameter roughly corresponding to the diameter of the coupling device, wherein the K-wire serves as a guide for said larger drill, after which the larger drill is removed and the coupling device is, in turn, introduced into the femoral neck by means of the K-wire as a guide.

It is particularly beneficial if a cannulated introduction device is provided which can be connected to the nail alternatively to the adjusting device, in particular can be inserted into the bore of the nail in place of the adjusting device, so that bone cement or another augmentation material can be introduced up to a region of a femoral head through the cannulated introduction device when the bone nail is arranged in a femoral shaft and the introduction device is connected to the nail. Typically, the bone cement or the other augmentation material is introduced through a cannula which is detachably and temporarily introduced into the cannulation of the introduction device that can be embodied as a through-bore through the introduction device. Typically, the apparatus also comprises a cannula of this type, which corresponds to the cannulated introduction device.

Of course, alternatively to a cannula which is temporarily introduced into the introduction device in order to introduce the bone cement or the other augmentation material into the first bone bore through the introduction device, it can also be provided that the introduction device itself is embodied as a cannula, so that the bone cement or the other augmentation material is directly introduced into the first bone bore through a bore in the introduction device and an additional, separate cannula is omitted.

It shall be understood that, alternatively to bone cement, any other suitable augmentation material can also be used. For the purposes of this application, the term bone cement therefore also comprises other suitable materials.

Thus, a method is rendered possible wherein the coupling device is introduced into the femoral neck in two stages, namely first only up to a position that is, for example, 20 mm to 40 mm, in particular approximately 30 mm, away from an end position in the femoral neck, after which bone cement is introduced into an end region of the femoral neck via the cannulated introduction device, typically using a cannula, whereupon the coupling device is moved into the end position in the femoral neck in order to displace the bone cement located in said end position, whereby the bone cement is distributed in the bone. This is very advantageous for patients with advanced osteoporosis in particular, in order to distribute the bone cement in the porous bone, and thus to stabilize the bone. Particularly preferably, the K-wire is once again only removed once the coupling device has been fully introduced into the femoral neck up to the end position, and typically also only once the bone cement has fully set. As a result, it is reliably prevented that bone cement advances to the joint capsule, which cannot be allowed to happen under any circumstances. This could happen, however, if the K-wire is removed sooner, especially since there is a risk that the femoral head will be pierced through during an introduction of the K-wire into the femoral head, so that a full-length opening all the way to the joint capsule is formed as a result. Put differently, the K-wire thus efficaciously closes a possible pass-through opening into the joint capsule during the introduction of the bone cement, whereby an unintended penetration of the bone cement into the joint capsule is prevented.

Preferably, the coupling device comprises at an end a thread with which the coupling device can be fixed in place in the femoral head. For an interaction of the coupling device with the adjusting device, the coupling device furthermore typically comprises a contact surface corresponding to the adjusting device, which contact surface can also be simultaneously embodied as a groove in order to prevent a rotation by means of the adjusting device. The corresponding contact surface must typically be arranged on a side on which the adjusting device is arranged relative to the coupling device, typically distally. To nevertheless ensure a high variability in terms of a possible screw-in depth, it is preferably provided that at least two corresponding contact surfaces are provided such that they are distributed over a circumference of the coupling device. With a thread pitch of 3.2 mm, for example, the coupling device can then be introduced into the femoral head not only in a 3.2-mm step screw-in depth, but rather, with two contact surfaces arranged such that they are uniformly distributed over the circumference for example, in a 1.6-mm step screw-in depth.

The adjusting device typically comprises a roughly cylindrical or conical outer contour with a thread, wherein the adjusting device can be screwed into the nail by means of the thread, typically at a position arranged distally relative to the coupling device. A length of the adjusting device is typically greater than a length of an opening in the nail, through which opening the adjusting device is guided through the nail, so that the adjusting device typically protrudes past the nail on both sides. It can also be provided that the adjusting device is embodied to be nearly as long as the coupling device, so that the adjusting device can project into a region of the femoral head from a lateral side of the nail. As a result, the coupling device can be stabilized and a rotation of the coupling device about the transverse axis as well as a rotation of a femoral head fragment about the coupling device can be effectively prevented.

According to the invention, the other object is attained by a method of the type named at the outset in which an adjusting device is connected to the nail, which adjusting device limits a mobility of the coupling device relative to the nail along a transverse axis. Typically, an apparatus according to the invention is used for a method of this type. The transverse axis is typically roughly transverse to a longitudinal axis of the nail, normally at an angle at which an axis of the femoral neck lies to an axis of the femoral shaft, for example at an angle of 110° to 140°.

It is beneficial if a first K-wire is first introduced into the femoral neck up to the femoral head along the transverse axis, which first K-wire has a smaller diameter than the coupling device, after which a first bone bore for the coupling device is introduced into the femoral neck, wherein the first K-wire serves as a guide, whereupon the coupling device is introduced into the femoral neck through the nail. This ensures a particularly precise introduction of the coupling device into the femoral neck, especially since the bore required for a K-wire has a very small diameter of 1.5 mm, for example, which bore can be introduced with high precision. Normally, the bore for this purpose is introduced by means of the K-wire, so that the K-wire initially serves as a drill and subsequently as a guide for the larger bone bore and the coupling device.

Typically, the K-wire also serves as a guide for the introduction of the coupling device into the femoral neck or into the femoral head.

Particularly preferably, it is provided that the K-wire is removed once the coupling device has been fully introduced into the femoral neck. The K-wire is thus only removed in a lateral direction after a final positioning of the coupling device in the femoral neck.

Particularly for fractures in the region of the base of the femoral neck, it can be advantageous to introduce a second K-wire into the femoral head with an axial offset, in order to prevent a rotation of the femoral head when drilling for or screwing-in the coupling device.

For osteoporosis patients in particular, it can be advantageous if the coupling device is initially only introduced into the first bone bore up to an intermediate position, after which bone cement is introduced into an end region of the first bone bore, whereupon the coupling device is fully introduced into the first bone bore, wherein a bone cement located in the end region is distributed in the femoral head. A concluding introduction of the coupling device into the end region thus causes a distribution of the bone cement, wherein the coupling device acts as a plunger which distributes the cement. The coupling device can thus be temporarily fixed in place up to approximately 30 mm before a final position in a first step, for example, after which the bone cement is introduced, whereupon the coupling device is moved a further 30 mm into the final end position in a medial direction. Following a setting of the bone cement, the K-wire is then removed. If the femoral head was pierced through during the introduction of the K-wire into the femoral head, it is, because of the K-wire located in the femoral head, thus prevented during the cementing that the bone cement passes from the femoral head into the joint capsule through the pass-through opening that may have been unintentionally created.

Particularly preferably, it is provided that the bone cement is introduced into the end region of the first bone bore through a cannulated introduction device, which introduction device is guided through the nail through an opening through which the adjusting device can be guided through the nail, and is removed from the nail after the introduction of the bone cement, whereupon the adjusting device is inserted, in particular screwed, into the opening. The opening can, as stated, coincide with the bore, so that in a bore that is embodied to be elongate, the coupling device is arranged in an upper, or proximal, region and the adjusting device in a lower, or distal, region in the bore.

To introduce the adjusting device, which can likewise project into the femoral head region, a second bone bore is typically introduced into the femoral neck, typically likewise from a lateral side of the femur. For this purpose, it is preferably provided that, distally from a region at which the coupling device can be connected to the nail, a second K-wire is introduced, in particular at an angle of 0.1° to 15°, in particular 0.8° to 2°, to the first K-wire, after which a second bone bore is introduced into the femoral neck, wherein the second K-wire serves as a guide, whereupon the second K-wire is removed, after which the adjusting device is introduced into the second bone bore, which adjusting device protrudes past the nail on both sides.

Additional features, advantages, and effects of the invention follow from the exemplary embodiments described below. In the drawings which are thereby referenced:

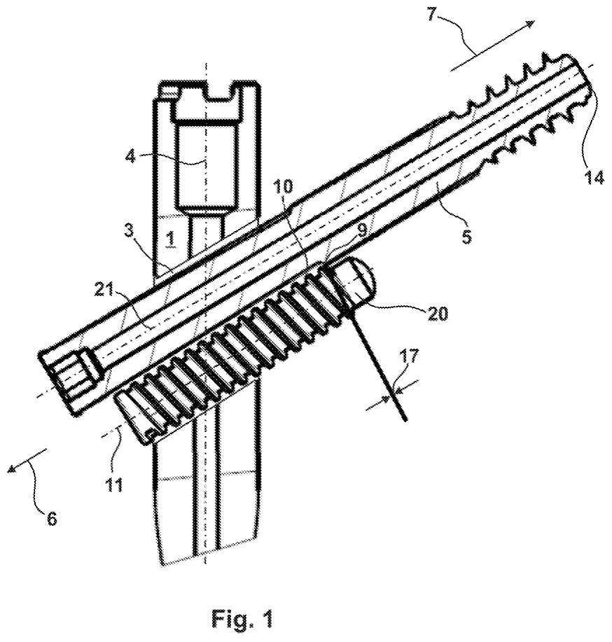

FIGS. 1 through 3 show an apparatus according to the invention in a sectional illustration and different working states;

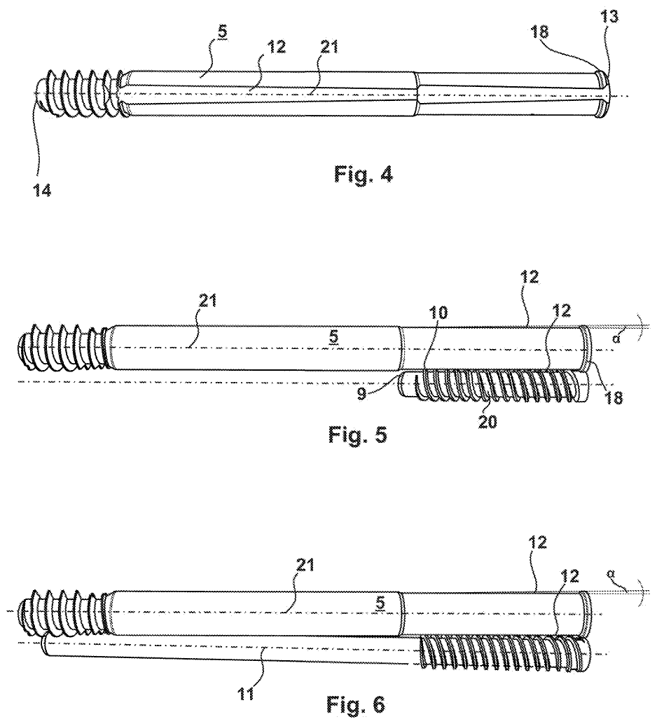

FIGS. 4 through 6 show detailed views of further apparatuses according to the invention;

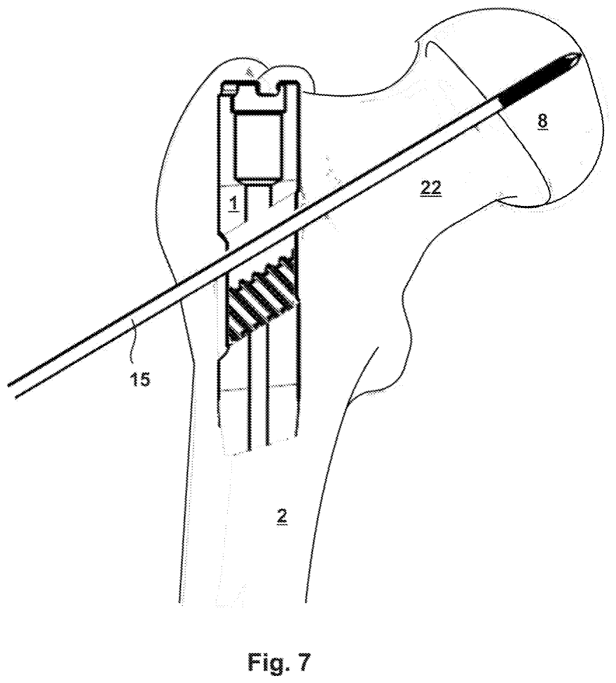

FIGS. 7 through 14 show different process steps for performing a method according to the invention;

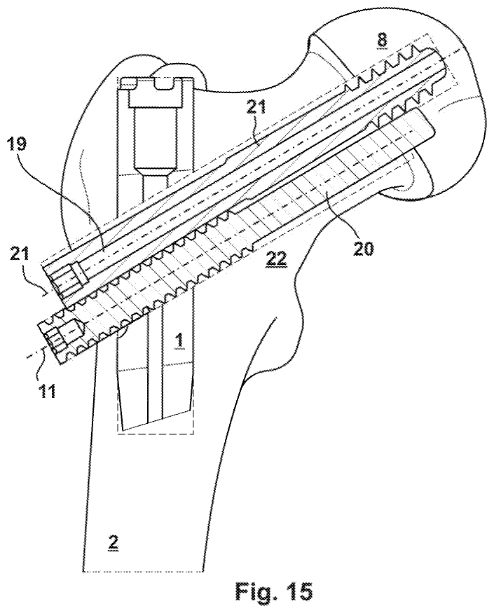

FIG. 15 shows a further apparatus according to the invention;

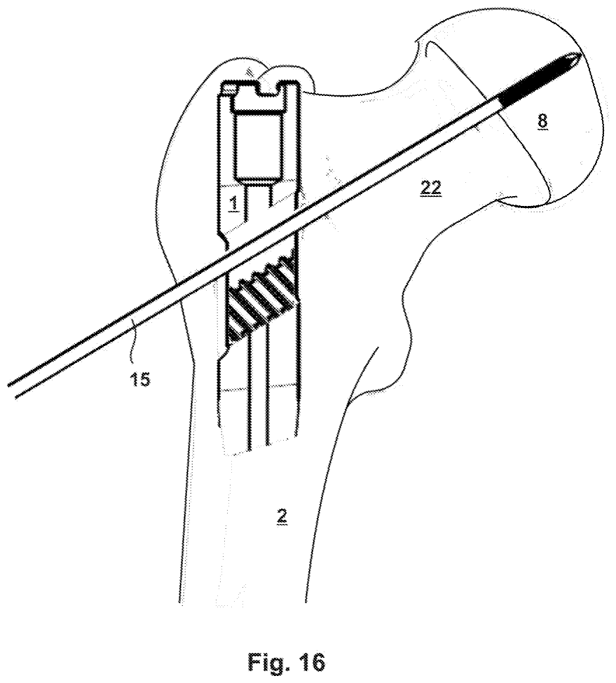

FIGS. 16 through 26 show different process steps in a further method according to the invention;

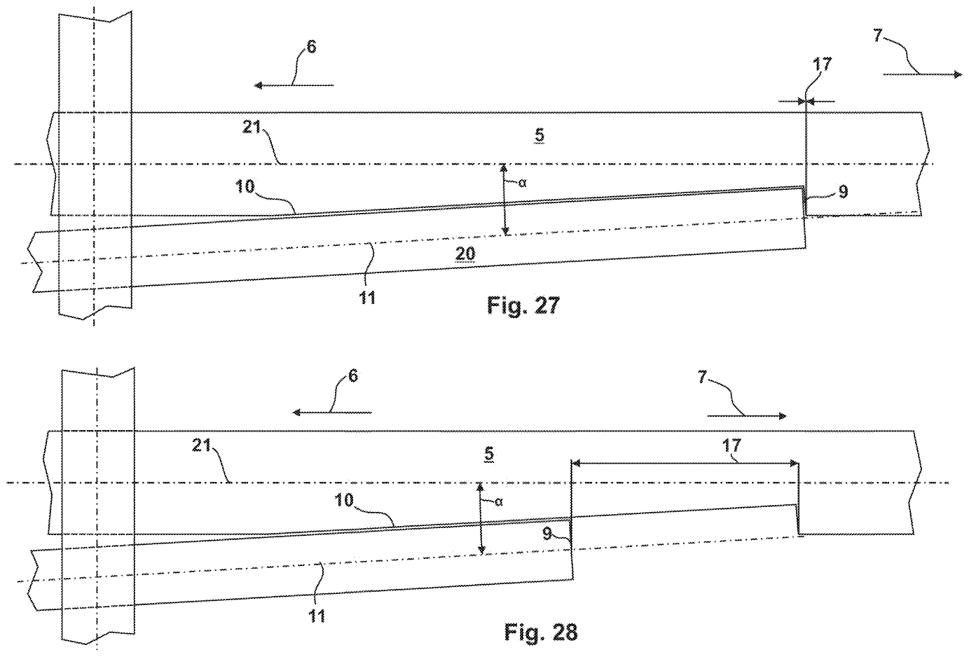

FIGS. 27 through 29 show a detailed view of an apparatus according to the invention in a schematic illustration:

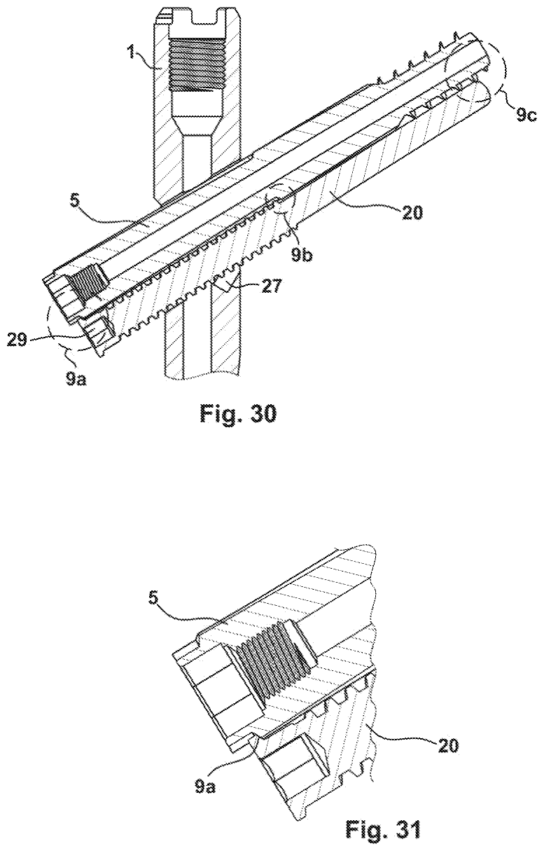

FIGS. 30 and 31 show detailed views of a further apparatus according to the invention.

FIGS. 1 through 3 show an apparatus according to the invention in different working states; The apparatus comprises a bone nail, only partially illustrated, which can be arranged along a longitudinal axis 4 in an intramedullary canal of a femur, which intramedullary canal begins at an upper, or proximal, end of the femur. In addition, the apparatus comprises a coupling device, embodied here as a bone screw 5, which is arranged in a bore 3 of the nail 1 in order to connect the femoral shaft 2 to a femoral head 8 that has broken off. For this purpose, a thread is provided at a medial end 14 of the bone screw 5, which thread can be screwed into the femoral head 8. The bore 3 and the bone screw 5 are thereby aligned such that the bone screw 5 can be connected to the nail 1 along a transverse axis 21 that is aligned transversely to the longitudinal axis 4 of the nail 1, typically at an angle of approximately 130° to the longitudinal axis 4 of the nail 1, so that the transverse axis 21 roughly extends along a femoral neck and the nail 1 roughly extends along the femoral shaft 2.

The bone screw 5 is not screwed together with the nail 1, but rather is connected such that it can be moved along a transverse axis 21, whereby a particularly good healing is achieved. Furthermore, in the apparatus according to the invention an adjusting device is provided, which in this case is embodied as an adjusting screw 20, that can be screwed together with the nail 1, namely in an opening which in this case is arranged in a lower region of the bore 3 through which the bone screw 5 runs through the nail 1.

Here, the adjusting screw 20 limits a mobility of the bone screw 5 relative to the nail 1, depending on a screw-in depth. For this purpose, a first stop surface 9 is provided on the one hand, which stop surface 9 limits a mobility of the bone screw 5 in a lateral direction 6. The first stop surface 9 on the adjusting screw 20 thus forms a lateral end position of the bone screw 5 and is oriented roughly perpendicularly to an axis of the adjusting screw 20 or an adjusting direction 11. The first stop surface 9 on the adjusting screw 20 can be formed by a thread flank, for example.

Furthermore, a second stop surface 10 is also provided on the adjusting screw 20, which second stop surface 10 limits a mobility of the bone screw 5 in a medial direction 7. The second stop surface 10 is arranged on a circumferential surface of the adjusting screw 20, and is therefore oriented roughly perpendicularly to the first stop surface 9 and roughly coaxially with the adjusting direction 11. This second stop surface 10 results from an angle α of approximately 1.2° between the adjusting direction 11 and the transverse direction.

In FIG. 1, a position of the adjusting screw 20 is illustrated in which said adjusting screw 20 is completely screwed into the nail 1, so that the bone screw 5 cannot be moved relative to the nail 1, but rather the bone screw 5 bears against both the first stop surface 9 and also against the second stop surface 10 on the adjusting screw 20.

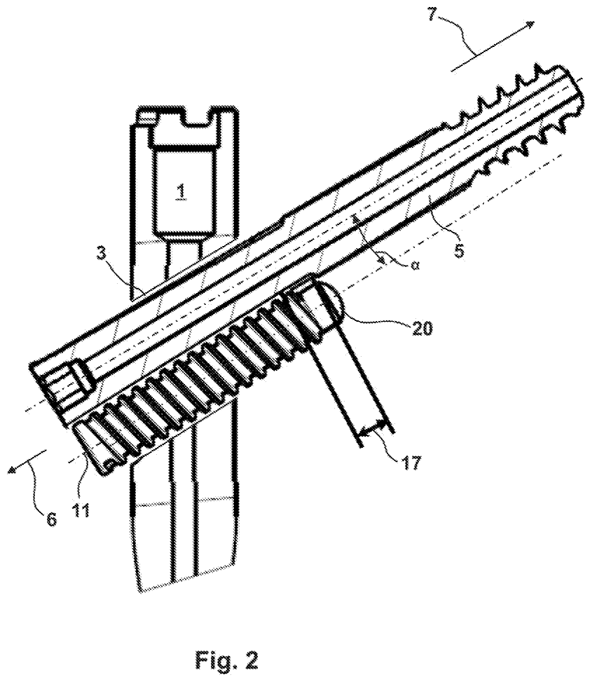

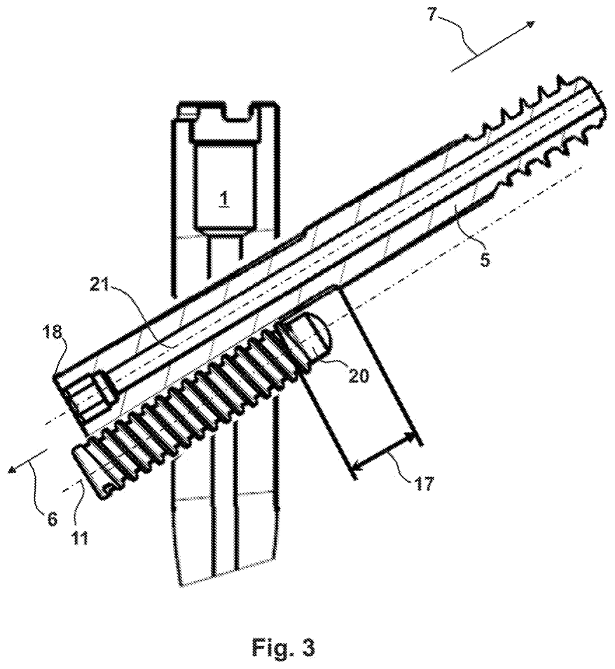

FIGS. 2 and 3 show further working positions, wherein in FIG. 2 compared to FIG. 1, the adjusting screw 20 has been slightly screwed out of the nail 1 in a lateral direction 6, whereby a mobility or a play 17 of the bone screw 5 relative to the adjusting screw 20 or relative to the nail 1 occurs in a lateral direction 6 and, oppositely, in a medial direction 7.

FIG. 3 shows a further working position, wherein the adjusting screw 20, compared to FIG. 2, has been screwed out of the nail 1 even further in a lateral direction 6, so that the play 17 of the bone screw 5 is larger than in the working position illustrated in FIG. 2.

A mobility of the bone screw 5 relative to the bone nail is thereby limited by the adjusting screw 20 in a lateral direction 6 using a first stop surface 9, which in this case is arranged at a medial end 14 of the adjusting screw 20. A mobility in the direction of the transverse axis 21 in a medial direction 7 is limited by a second stop surface 10 on the adjusting screw 20 that is formed by a roughly cylindrical outer surface of the adjusting screw 20. This results from an angle & at which an adjusting axis of the adjusting screw 20 is oriented to the transverse axis 21, so that a movement of the adjusting screw 20 along the adjusting direction II has different effects on positions of the first contact surface and second contact surface. Thus, with a movement of the adjusting screw 20 along the adjusting direction 11, not only is a position of the stop surfaces displaced, but rather a play 17 of the bone screw 5 relative to the nail 1 and relative to the adjusting screw 20 also changes. The angle and dimensions of the bone screw and adjusting screw can thereby also be chosen such that a position of the second stop surface, or a medial end position of the bone screw relative to the nail, is not altered by a change in the position of the adjusting screw, but rather a change in the screw-in depth only affects the position of the first stop surface.

FIGS. 4 through 6 show further exemplary embodiments of the coupling device and adjusting device of apparatuses according to the invention. Here, the coupling device is also respectively embodied as a bone screw 5 with a medial thread, and the adjusting device is respectively embodied as an adjusting screw 20. Particularly in FIG. 4, a groove 12 corresponding to the first contact surface and second contact surface of the adjusting screw 20 is also visible along a longitudinal direction on the bone screw 5, which groove 12 on the one hand provides contact surfaces on the bone screw 5 that correspond to the first contact surface and second contact surface and on the other hand prevents a rotation of the bone screw 5 about the transverse axis 21 when the adjusting screw 20 is arranged in the region of said contact surface. For this purpose, the groove 12 corresponds to the adjusting screw 20 and, in the working position, is preferably roughly parallel to the adjusting direction 11. Consequently, the groove 12 typically lies at an angle a of 1.2° to the transverse direction 21, for example.

The corresponding groove 12, which typically comprises surfaces corresponding to the first stop surface 9 and to the second stop surface 10, may be provided only once on the bone screw 5, typically roughly along an axis of the bone screw 5 or the transverse axis 21. It is preferably provided, however, that said groove 12 is arranged multiple times, at least twice, such that it is distributed over a circumference of the bone screw 5, as illustrated in FIG. 5 and FIG. 6, especially since there are then multiple possibilities for how the bone screw 5 can be screwed into the nail 1. Thus, with a thread pitch of 3.2 mm for example, a screw-in depth in 1.6-mm steps is possible if the corresponding groove 12 is arranged such that it is distributed over the circumference at two opposing positions, especially since one of the grooves 12 is, in the working position, advantageously arranged at a position corresponding to the adjusting screw 20. As can be seen in FIG. 5 and FIG. 6, the transverse axis 21 and the adjusting axis lie at an angle u to one another, whereby a ramp results which leads to the different effect of the movement of the adjusting device 20 in the adjusting direction 11 on the individual first stop surface 9 and the second stop surface 10.

As can be seen in FIG. 5 and FIG. 6, the adjusting screw 20 can also be embodied as a long screw that projects into a region of the femoral head 8 in order to effectively prevent a rotation of the femoral head 8 about the bone screw 5 when placed under strain.

As can be seen in the exemplary embodiments of FIGS. 4 through 6, the bone screws 5, according to these embodiments, respectively comprise at a lateral end 13 a collar 18 that can serve as a stop and can define a medial end position of the bone screw 5 in an interaction with the adjusting screw 20 and/or the nail 1. This collar 18 can also be omitted, however, especially since a medial migration can also be defined by an interaction of the bone screw 5 and adjusting screw 20.

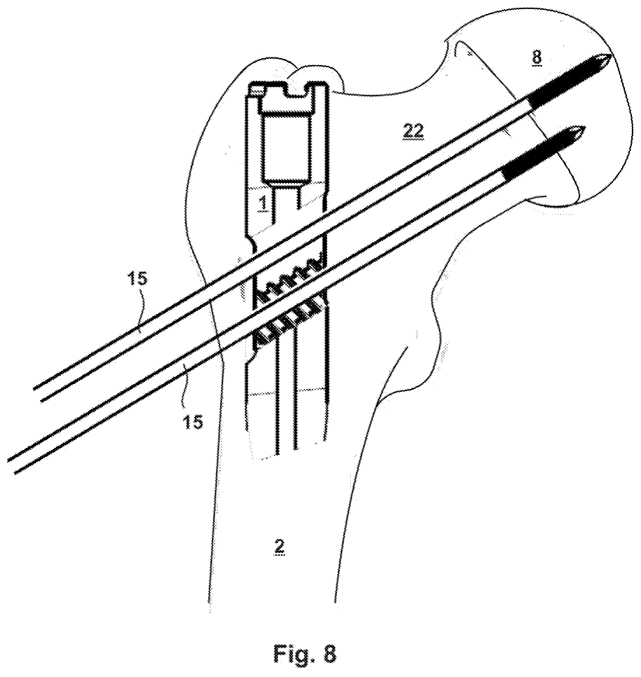

FIGS. 7 through 14 show different steps of a method according to the invention for treating a femoral fracture. As can be seen in FIG. 7 and FIG. 8, K-wire bores are first introduced into the femoral neck 22 using a drill with a small diameter, in which bores K-wires 15 are then positioned. For this purpose, the K-wires themselves are typically embodied as drills so that the K-wire bores can be introduced using the K-wires and the K-wires 15 then remain in the bone. The K-wires 15 are then used in order to guide drills with larger diameters, so that a first bone bore 23 for the coupling device, in this case also embodied as a bone screw 5, and a second bone bore 24 for the adjusting device, in this case also embodied as an adjusting screw 20, can be introduced in a precise manner.

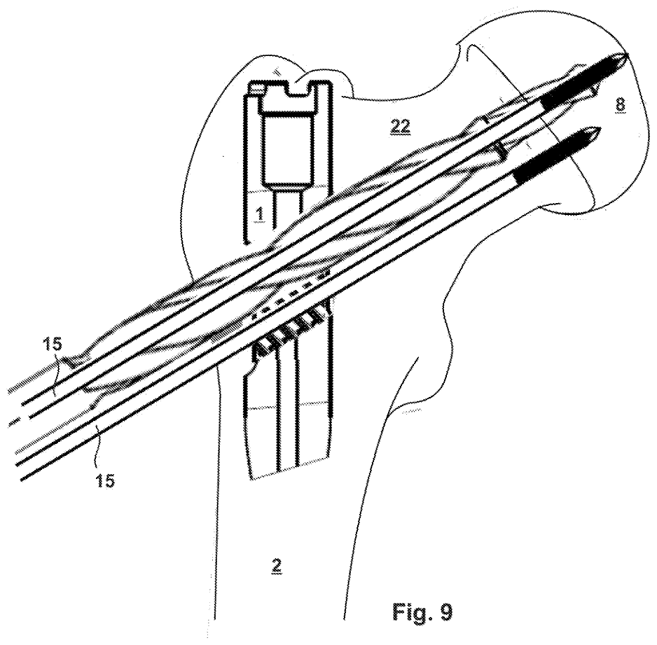

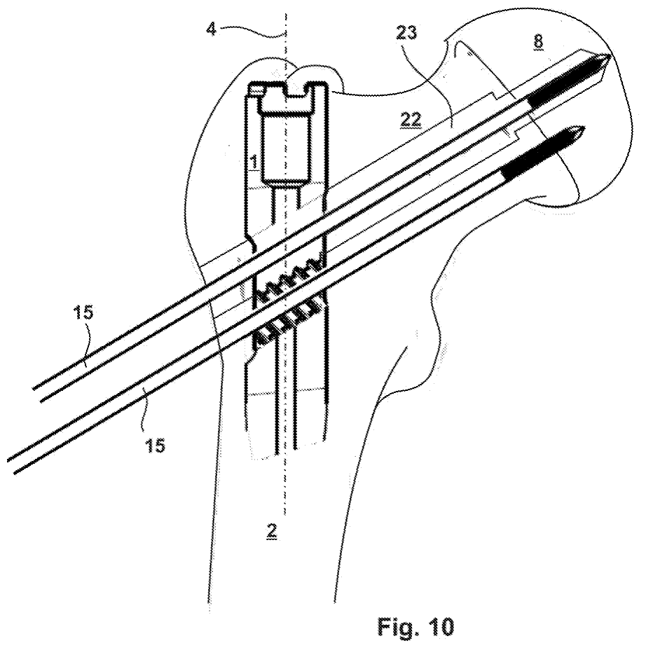

FIG. 7 thereby shows a situation in which a first K-wire has been introduced. FIG. 8 shows a method step in which the second K-wire 15 has been introduced. FIG. 9 shows a method step in which a first bone bore 23 for the bone screw 5 is introduced into the femoral neck 22, wherein a drill is embodied to be hollow and is guided by the corresponding K-wire 15. For this purpose, an additional positioning aid can also be provided, for example by means of X-raying or the like. FIG. 10 shows a situation in which the first bone bore 23 has already been formed, but the second bone bore 24 has not yet been formed. As can be seen, the K-wire 15 remains in the first bone bore 23 even after the drilling of the first bone bore 23.

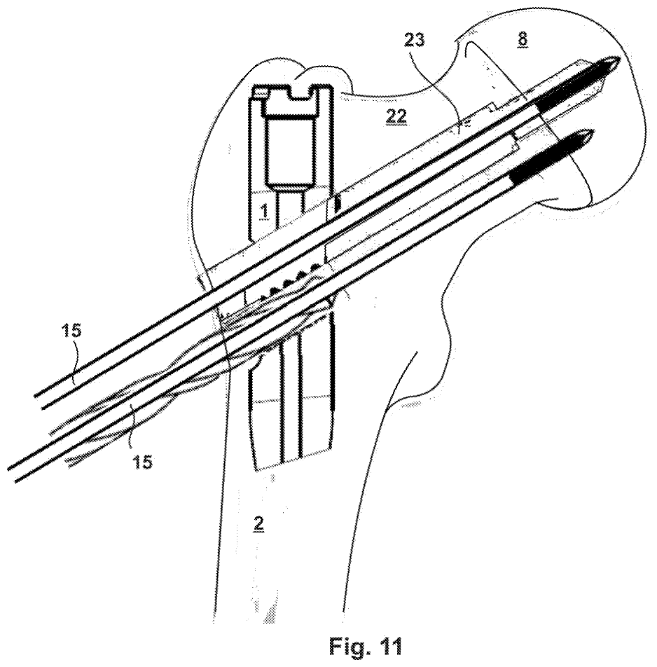

FIG. 11 shows a method step in which the second bone bore 24 is drilled, once again with the use of a K-wire 15 as a guide.

Alternatively to the use of a K-wire 15 as a guide, the second bone bore 24 can also be introduced without K-wire 15, that is, without a guide, and using a solid drill.

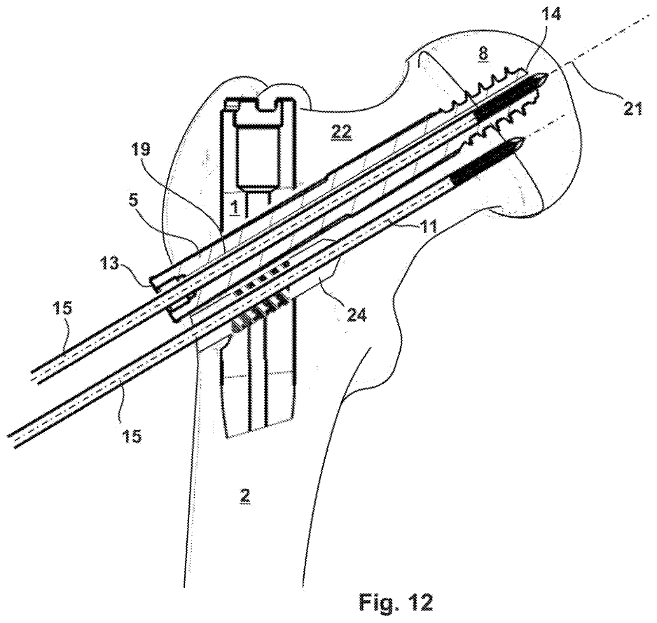

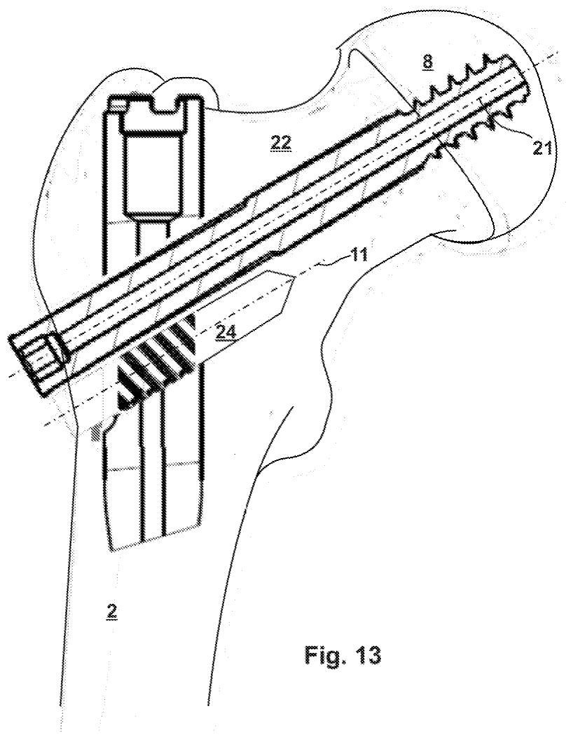

FIG. 12 shows a method step, wherein the second bone bore 24 has also already been completed and the bone screw 5 is introduced into the first bone bore 23, namely likewise by means of the K-wire 15, in that the K-wire 15 serves as a guide for the bone screw 5. For this purpose, the bone screw 5 comprises a central through-bore 19 that extends from a medial end 14 to a lateral end 13. The K-wire 15 is therefore only removed once the bone screw 5 has been fully introduced into the first bone bore 23. The second K-wire 15 is then also removed, as can be seen in FIG. 13.

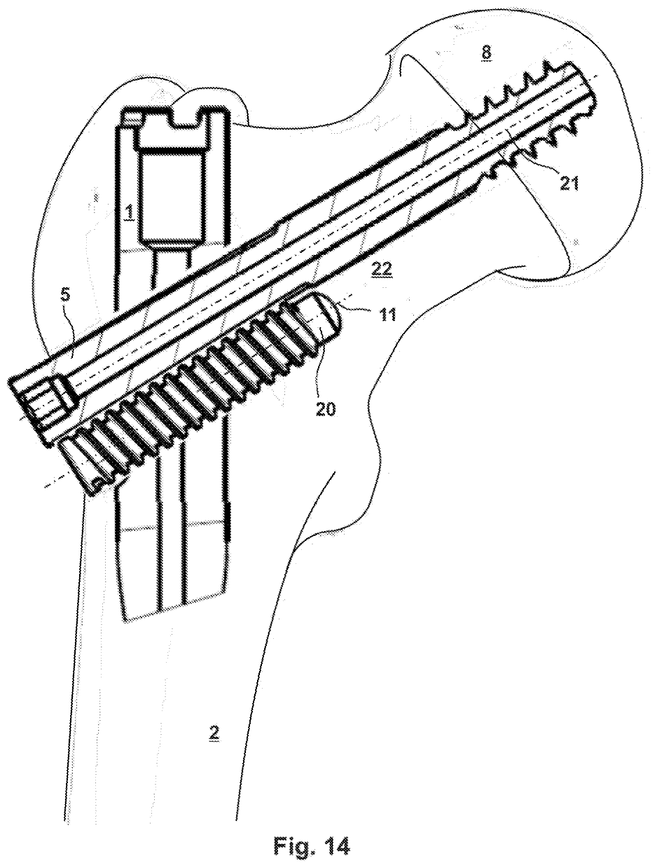

Finally, the adjusting screw 20 is introduced, as can be seen in FIG. 14. Using a position of the adjusting screw 20 along the adjusting direction 11, which position can be altered by means of the thread on the adjusting screw 20 and the nail 1, a mobility of the bone screw 5 relative to the nail 1 can be influenced.

FIG. 15 shows an alternative embodiment, wherein a long adjusting screw 20 is provided which projects into a region of the femoral head 8. In an embodiment of this type, a rotation of the femoral head 8 about the bone screw 5 is prevented in an effective manner. The adjusting screw 20 thus also serves as an anti-rotation mechanism for the femoral head 8 relative to the femoral shaft 2.

FIGS. 16 through 26 show a further method according to the invention for treating a femoral fracture, wherein a bone cement 26 is also introduced into the femoral head 8.

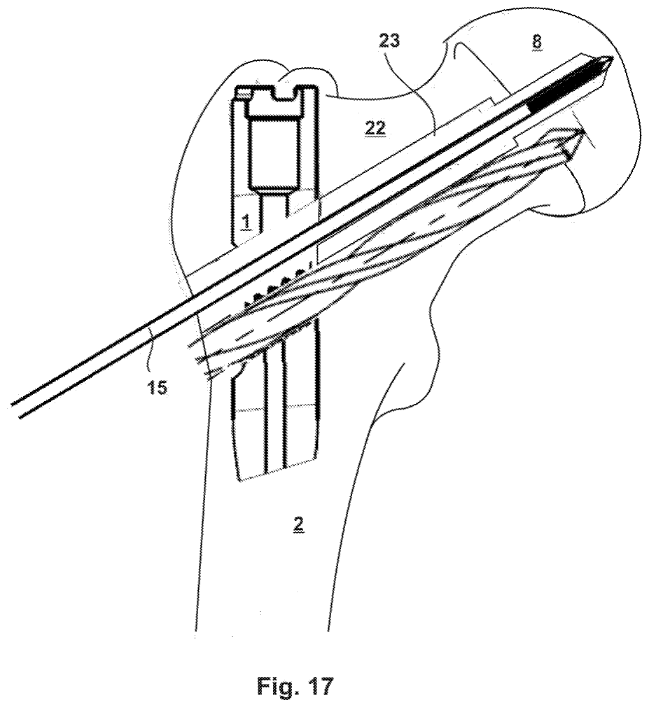

As depicted in FIGS. 16 and 17, a first bone bore 23 is again introduced into the femoral neck 22 for this purpose, wherein in this case a K-wire 15 is once again introduced beforehand, which K-wire 15 subsequently serves as a guide for the larger drill.

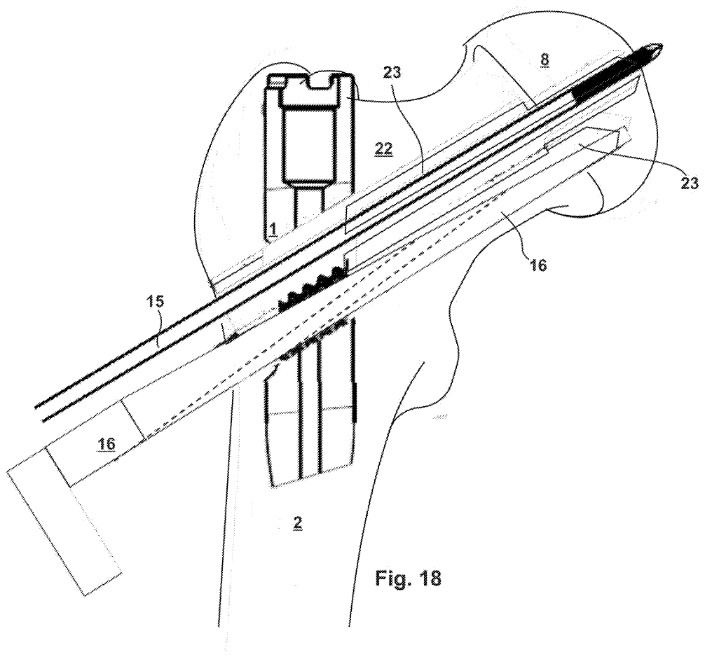

The method illustrated here relates to the treatment of a femoral fracture on an osteoporosis patient, for which reason bone cement 26 is introduced prior to a final introduction of the bone screw 5 and adjusting screw 20 into an end region of the femoral head 8. For this purpose, an introduction device 16 is provided which is illustrated in FIGS. 18 through 23. The introduction device 16 is introduced into the femoral neck 22 through an opening into the nail 1 and into the second bone bore 24, through which opening the adjusting screw 20 is alternatively, and also subsequently, introduced through the nail 1. In this case, this opening is also part of the bore 3 through which the bone screw 5 is guided through the nail 1.

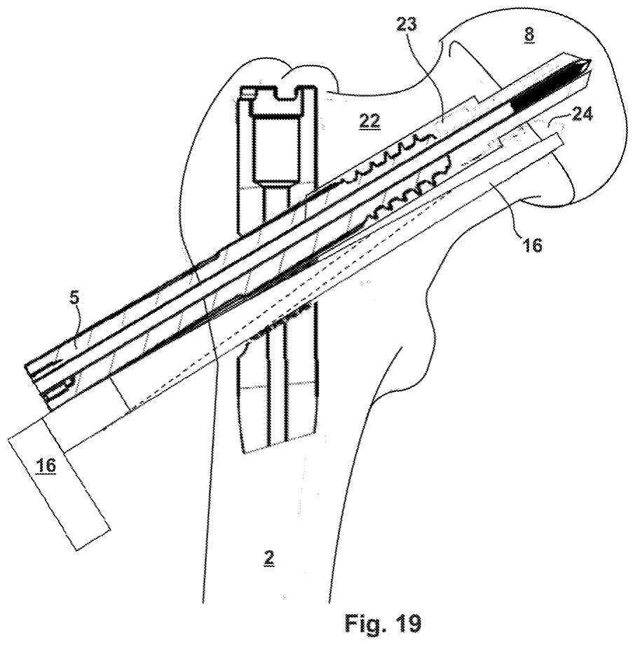

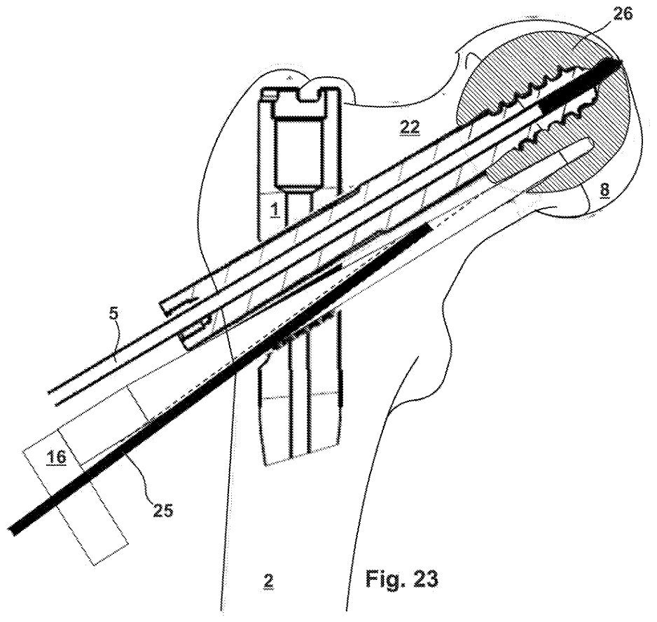

The illustrated introduction device 16 thus comprises, at least in a region in which said device is in contact with the nail 1, an outer contour corresponding to the adjusting screw 20, but no thread, even though this would be possible, of course. The introduction device 16 is furthermore cannulated, so that once said device has been positioned under the bone screw 5, bone cement 26 can be introduced into the end region of the first bone bore 23 through the cannulation by means of a cannula 25. For this purpose, the bone screw 5 is initially not fully introduced into the first bone bore 23, but rather, as can be seen in FIGS. 19 through 22, only up to an introduction position, which introduction position is, for example, 30 mm away from an end position in which the bone screw 5 is not located until in the method step illustrated in FIG. 23.

While the bone screw 5 is held at this introduction position illustrated in FIGS. 19 through 22, bone cement 26 is introduced into the end region of the first bone bore 23, or into a region of the femoral head 8, by means of a cannula 25 that is inserted into the introduction device 16.

Alternatively to a cannula 25 that is temporarily inserted into the introduction device 16, the introduction device 16 itself can also be embodied as a cannula 25.

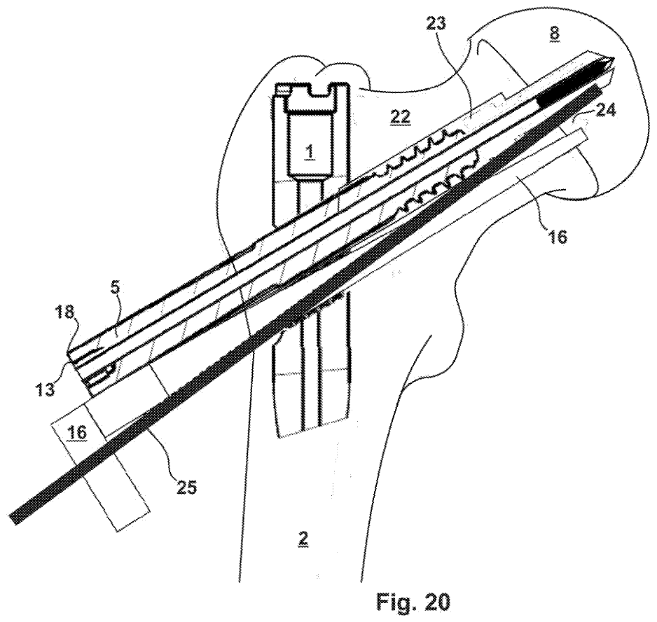

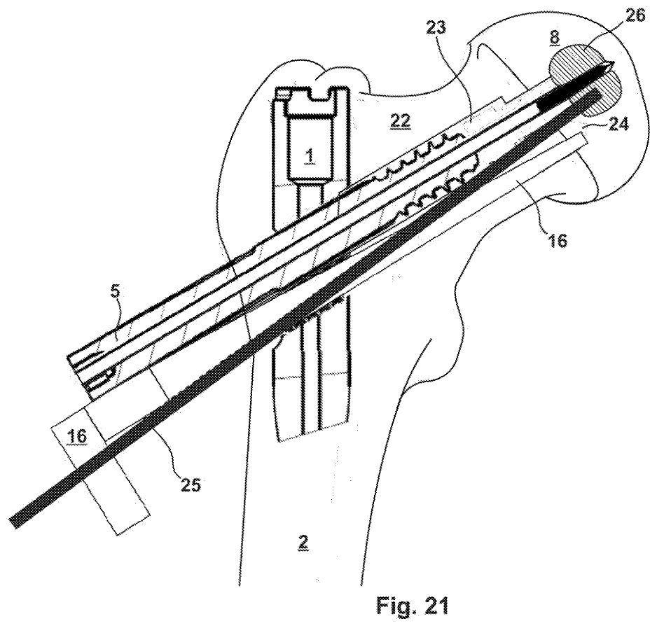

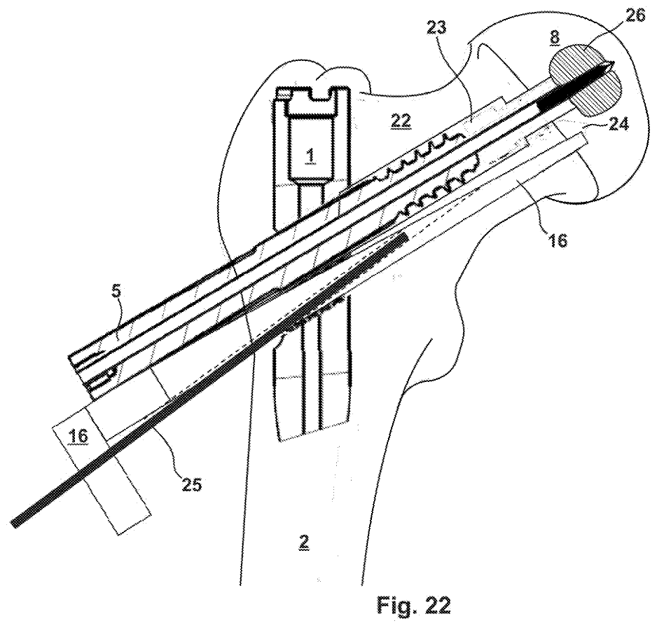

An introduction of the cannula 25 into the cannulation of the introduction device 16 that is embodied as a bore is illustrated in FIG. 20, and the introduction of the bone cement 26 into the femoral head 8 through the cannula 25 is illustrated in FIG. 21. FIG. 22 shows the removal of the cannula 25 from the introduction device 16 while the introduction device 16 remains in the second bone bore 24.

The bone screw 5 is subsequently fully screwed into the end position in the first bone bore 23. namely prior to a setting of the bone cement 26, wherein the bone cement 26 located in the end position and having not set is distributed in the femoral head 8, as illustrated in FIG. 23.

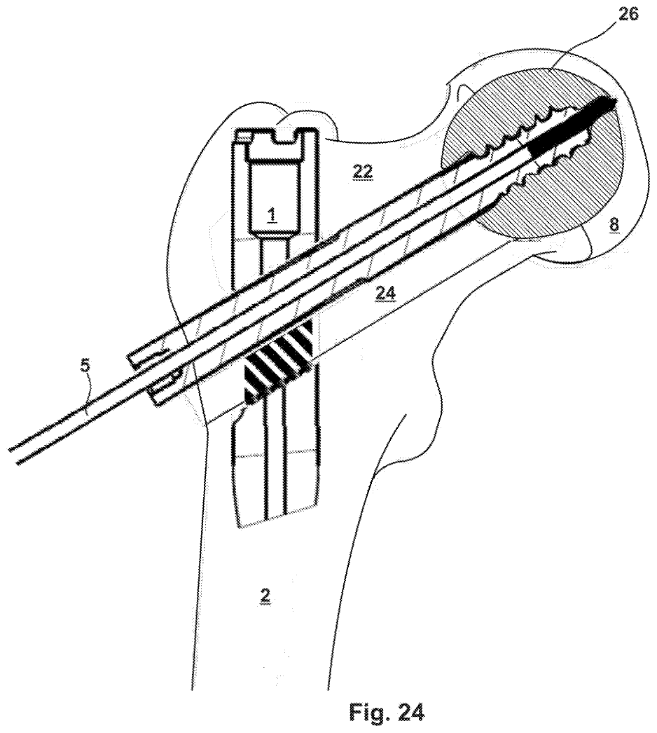

The introduction device 16 is then removed from the second bone bore 24 and the bore 3 of the nail 1, as can be seen in FIG. 24.

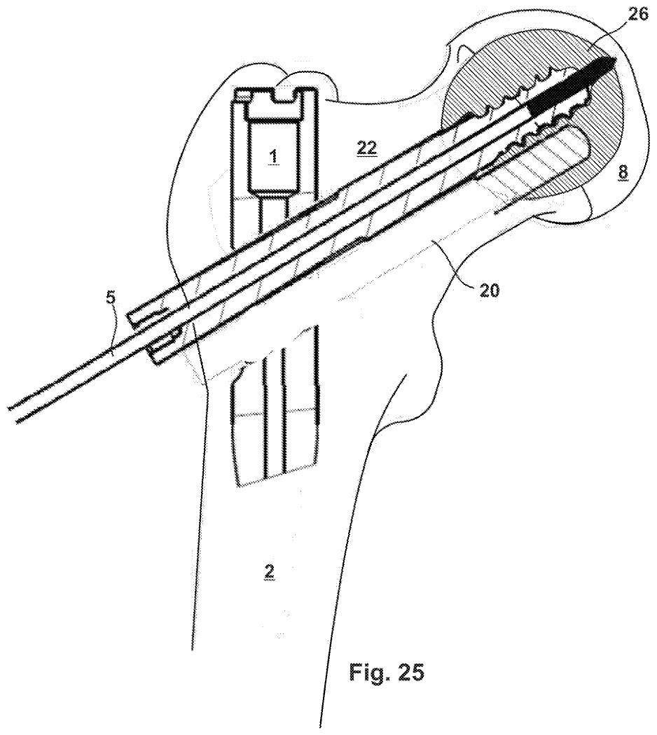

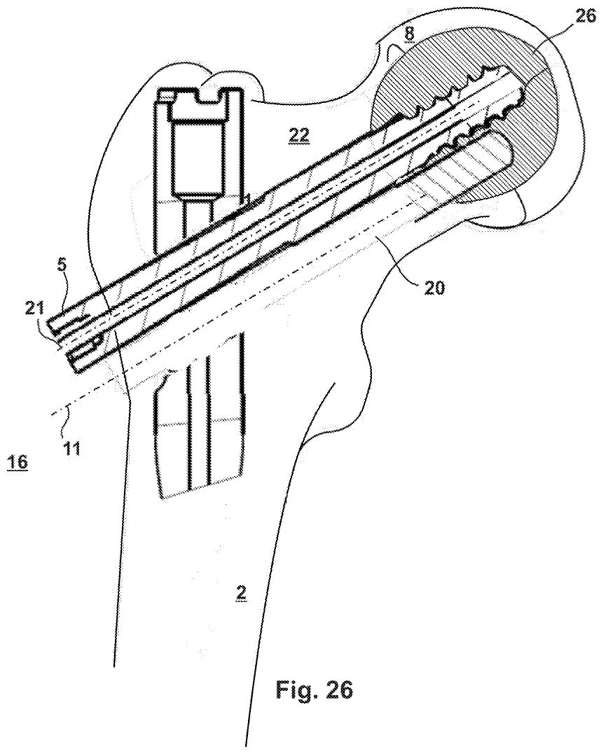

As can be seen in FIG. 25, the adjusting screw 20 is subsequently screwed into the nail 1 distally from, or below, the bone screw 5 and ultimately, once the bone cement 26 has set, the K-wire 15 is removed from the bone screw 5, which K-wire 15 guides the bone screw 5 during an introduction. This position, which constitutes a completion of the introduction of the apparatus for treating the fracture, is illustrated in FIG. 26. The depicted adjusting screw 20 reaches into the region of the femoral head 8, so that said screw not only allows an ability to set the play 17 of the bone screw 5 and prevents a rotation of the bone screw 5 about the transverse axis 21, but rather also inhibits a rotation of the femoral head 8 about the transverse axis 21 and thus a loosening of a screw connection between the femoral head 8 and bone screw 5.

By not removing the K-wire 15 from the first bone bore 23 until after the bone cement 26 has set, the liquid bone cement 26 is effectively prevented from penetrating into the joint capsule before setting if the femoral head 8 is unintentionally pierced through by the K-wire 15 during an introduction of the same, especially since the K-wire 15 seals a possible unintentionally caused opening in the femoral head 8.

A collar 18 can also be provided at a lateral end 13 of the bone screw 5 in this embodiment, in order to define a medial end position through a form fit with the nail 1 and/or the adjusting screw 20 or a stop.

An apparatus according to the invention enables a particularly effective treatment of a femoral fracture, especially since the mobility of the bone screw 5 relative to the nail 1, and therefore a mobility of the femoral head & relative to the femoral shaft, can also easily be altered while an operation is still in progress, in order to be able to adapt the mobility particularly well to the individual patient.

FIGS. 27 through 29 schematically show an interaction of the bone screw 5 and adjusting screw 20 in an embodiment of an apparatus according to the invention similar to the situations depicted in FIGS. 1 through 3, but in this case with a detailed illustration of the interaction of a first contact surface, second contact surface, and groove 12. Here, in order to visualize the interaction particularly well, a variant is furthermore shown with an angle α of approximately 3°, a groove 12 extending nearly to the transverse axis 21, and a nail 1 having a, in comparison with the bone screw 5 and adjusting screw 20, very small diameter, whereas in actual apparatuses, the angle α is typically less than 2°, the groove 12 can also be less markedly pronounced, and the diameters of the nail 1, bone screw 5, and adjusting screw 20 can also differ from those illustrated in FIG. 27 through FIG. 29, as is illustrated in particular in FIGS. 1 through 26.

As can be seen, the groove 12 in the bone screw 5 corresponds to the adjusting screw 20, namely both in terms of dimensions and also in terms of an angle a at which the groove 12 and the adjusting screw 20 lie to the transverse axis 21.

If, as in this case, the adjusting screw 20 is formed by a body that is essentially cylindrical in the region of the groove 12 and the groove 12 is embodied correspondingly, a cylinder envelope surface of the adjusting screw 20, which is rotationally symmetrical with the adjusting axis, forms a second stop surface 10 in interaction with a corresponding surface of the groove 12, namely already starting at a screw-in depth of the adjusting screw 20 in the nail 1, at which screw-in depth the adjusting screw 20 contacts the bone screw 5. This second contact surface thus defines a medial end position of the bone screw 5, starting at which further movement of the bone screw 5 relative to the nail 1 in a medial direction 7, parallel to the transverse axis 21, is no longer possible, namely independently of a possible further movement of the adjusting screw 20 along the adjusting direction 11. Put differently, a position of the second stop surface 10 no longer changes starting from a contact of the adjusting screw 20 with the bone screw 5. Thus, the medial end position of the bone screw 5 is fixed starting from a contact of the adjusting screw 20 with the bone screw 5 in this exemplary embodiment, and the medial end position does not change with a screw-in depth of the adjusting screw 20, wherein this adjusting screw 20 is moved along the adjusting direction 11 if there is a change in the screw-in depth. A position of the second stop surface 10 is, in this exemplary embodiment, therefore constructively fixed via dimensions of the adjusting screw 20 and bone screw 5, as well as the angle a between the bone screw 5 and adjusting screw 20.

In contrast to the second stop surface 10, however, a position of the first stop surface 9 can be directly altered by a change in the screw-in depth of the adjusting screw 20. The first stop surface 9 defines a lateral end position of the bone screw 5, that is, a position starting at which further movement of the bone screw 5 in a lateral direction 6 relative to the nail 1 is no longer possible. This first stop surface 9 is, in this case, formed by a face of the adjusting screw 20 that is aligned roughly perpendicularly to the adjusting axis. The first stop surface 9 can, of course, also alternatively be formed by a different surface of the adjusting screw 20, the position of which is changed by a change in the screw-in depth relative to a corresponding surface of the groove 1, in particular with a screw flank

FIG. 27 shows a position of the apparatus analogous to that illustrated in FIG. 1, wherein a screw-in depth of the adjusting screw 20 is at a maximum, so that the bone screw 5 simultaneously bears against both the first stop surface 9 and also the second stop surface 10 and there is no play 17; the bone screw 5 therefore cannot be moved relative to the nail 1.

FIG. 28 shows a position analogous to the position illustrated in FIG. 2 or 3 in which the adjusting screw 20 has been laterally slightly screwed out of the nail 1; the screw-in depth is thus smaller than in the position illustrated in FIG. 27. The position of the bone screw 5 is unchanged from the position illustrated in FIG. 27. Because the position of the second stop surface 10, as stated, is not altered with the screw-in depth in this case, the bone screw 5 still bears against the second stop surface 10 and is thus still located in a medial end position in which no further movement of the bone screw 5 in the medial direction 7 is possible. However, the first stop surface 9 now no longer bears against the bone screw 5, as a consequence of which the bone screw 5 can be moved relative to the nail 1 along the transverse axis 21 in a lateral direction. Accordingly, there results a play 17, as illustrated, between the medial end position in which the bone screw 5 is located in FIG. 28 and a lateral end position up to which the bone screw 5 could be moved.

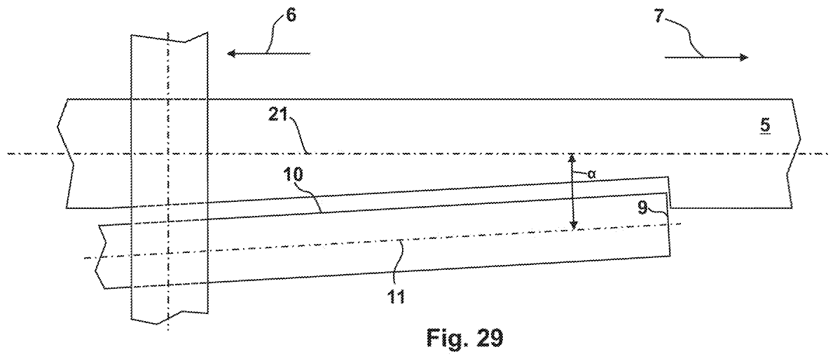

FIG. 29 shows the apparatus from FIGS. 27 and 28 with a screw-in depth unchanged from FIG. 28. In contrast to FIG. 28, however, the bone screw 5 is now located in a lateral end position, and thus bears against the first stop surface 9 on the adjusting screw 20, but no longer against the second stop surface 10. Accordingly, starting from this position, only a movement of the bone screw 5 relative to the nail 1 in a medial direction 7 is possible, but not any further movement in a lateral direction 6.

Here, a change in the screw-in depth thus acts with a cosine of the angle α, for small angles a therefore close to 1:1, on a change in the position of the first stop surface 9, but not on a position of the second stop surface 10. Accordingly, a size of the play 17 is also altered nearly 1:1 with the screw-in depth.

FIGS. 30 and 31 show detailed views of a further exemplary embodiment of an apparatus according to the invention. This apparatus comprises a first contact surface 9 formed by three stops that act in parallel, namely a primary stop 9a at a lateral end 13 of the adjusting device, also formed here by an adjusting screw 20, a secondary stop 9b at a medial end 14 of a thread of the adjusting screw 20, with which thread the adjusting screw 20 is screwed into the nail 1, and a tertiary stop 9c at a medial end 14 of the adjusting screw 20. At the tertiary stop 9c, a medial end 14 of the adjusting screw 20 interacts with a thread pitch of the bone screw 5. With the three stops 9a, 9b, 9c acting in parallel, an unintended sliding of the adjusting screw 20 along the bone screw 5 is prevented in a simple manner, and a mobility is thus reliably limited.

Additionally, in this exemplary embodiment a screw lock is provided which is formed by a plastic piece 27 in a thread pitch in the nail 1. This plastic piece 27 increases the friction between the thread of the adjusting screw 20 and the nail 1, so that an unintended loosening of the adjusting screw 20 is prevented.

Here, the adjusting screw 20 comprises a hexagon socket 29 at a lateral end 13 and can be actuated using a hex key, not illustrated. In order to prevent an unintentionally far screwing-in of the adjusting screw 20, the hex wrench is embodied such that it can only be guided up to a predefined position relative to the bone screw 5 in the medial direction 7 along the adjusting screw 20. For this purpose, a stop can be provided on the hex wrench and/or the bone screw 5, for example. Thus, starting at a predefined screw-in depth of the adjusting screw 20, the hex key loses contact with the adjusting screw 20, or slides out of the hexagon socket 29, so that no further screwing-in is possible. In this manner, an unintentionally far screwing-in of the adjusting screw 20 is prevented in a constructively simple design.

FIG. 31 shows the primary stop in a detailed view. As can be seen, the adjusting screw 20 comprises, at a lateral end, a collar formed by an adjusting screw collar 28, which collar bears against the bone screw 5 starting at a predefined relative position of the adjusting screw 20 and bone screw 5 and thus prevents a further movement of the bone screw 5 relative to the adjusting screw 20 in a lateral direction 6, so that this primary stop acts in parallel with the secondary stop 9b and in parallel with the tertiary stop 9c, as a result of which a sliding of the adjusting screw 20 on the bone screw 5 is effectively prevented.

In addition, a method according to the invention ensures that no bone cement 26 penetrates into the joint capsule, whereby a risk of complications is particularly low.

1. An apparatus for treating a fracture, comprising a nail which can be introduced into an intramedullary canal of a bone, wherein the nail comprises a bore that is aligned roughly transversely to a longitudinal axis of the nail, wherein a coupling device is provided which can be introduced into the bore such that the coupling device protrudes past the bore on both sides and can be moved within the bore along a transverse axis in a lateral direction and a medial direction oriented in an opposing manner to the lateral direction and which coupling device can be fixed in place at an end in a bone part, wherein an adjusting device is provided which can be fixed in place in different positions relative to the nail, and wherein the adjusting device comprises a first stop surface that limits a mobility of the coupling device in a lateral direction when the coupling device and adjusting device are arranged on the nail, so that by changing the position of the adjusting device relative to the nail, a lateral end position up to which the coupling device can be moved relative to the nail in a lateral direction can be altered.

2. The apparatus according to claim 1, wherein the first stop surface is at least partially formed by a shoulder or a collar in a region of a lateral end of the adjusting device.

3. The apparatus according to claim 1, wherein the first stop surface is at least partially formed by a part of a thread of the adjusting device, with which thread the adjusting device can be fixed in place relative to the nail.

4. The apparatus according to claim 1, wherein the first stop surface is at least partially formed by a part of a thread of the coupling device, with which thread the coupling device can be fixed in place at an end in a bone part.

5. The apparatus according to claim 1, wherein the adjusting device is connected to the nail by a thread, wherein a screw lock that increases a friction in the thread is provided.

6. The apparatus according to claim 1, wherein the adjusting device can only be screwed into the nail up to a predefined position, wherein this is constructively implemented with a wrench which, starting at a predefined position of the adjusting device, no longer produces any contact with a corresponding counterpiece in the adjusting device.

7. The apparatus according to claim 1, wherein the adjusting device comprises a second stop surface which, when the coupling device and adjusting device are arranged on the nail limits a mobility of the coupling device in a medial direction.

8. The apparatus according to claim 1, wherein a position of the adjusting device relative to the nail can be altered along an adjusting direction.

9. The apparatus according to claim 8, wherein the first stop surface and the second stop surface are aligned at different angles to the adjusting direction so that, when the position of the adjusting device relative to the nail is changed, the lateral end position can be changed by a greater magnitude than the medial end position.

10. The apparatus according to claim 1, wherein a position of the adjusting device relative to the nail can be altered along an adjusting direction, wherein the adjusting direction is aligned at an angle of 0.1° to 15°, to the transverse direction.

11. The apparatus according to claim 1, wherein the adjusting device is embodied to be longer than the bore so that the adjusting device can be connected to the nail in a position protruding past the bore on both sides, wherein the first stop surface is arranged medially from the nail.

12. The apparatus according to claim 1, wherein the coupling device comprises a groove that interacts with the adjusting device and runs along the coupling device, so that a rotation of the coupling device about the transverse axis is limited by the adjusting device when the coupling device and the adjusting device are connected to the nail.

13. The apparatus according to claim 1, wherein the bore is embodied to receive the coupling device in a proximal region and to receive the adjusting device in a distal region.

14. The apparatus according to claim 1, wherein the coupling device comprises a through-bore projecting from a lateral end to a medial end, into which through-bore a K-wire can be inserted.

15. The apparatus according to claim 1, wherein a cannulated introduction device is provided which can be connected to the nail so that bone cement can be introduced up to a region of a femoral head through the cannulated introduction device when the bone nail is arranged in a femoral shaft and the introduction device is connected to the nail.

16. A method for treating a fracture of the femoral neck, wherein a nail is introduced into an intramedullary canal of a femoral shaft after which a coupling device is introduced into the nail, which coupling device is fixed in place in a femoral head, wherein an adjusting device is connected to the nail, which adjusting device limits a mobility of the coupling device relative to the nail along a transverse axis.

17. The method according to claim 16, wherein a first K-wire is first introduced into the femoral neck up to the femoral head along the transverse axis, which first K-wire has a smaller diameter than the coupling device, after which a first bone bore for the coupling device is introduced into the femoral neck, wherein the first K-wire serves as a guide, whereupon the coupling device is introduced into the femoral neck through the nail.

18. The method according to claim 16, wherein the K-wire is removed once the coupling device has been fully introduced into the femoral neck.

19. The method according to claim 18, wherein the coupling device is initially only introduced into the first bone bore up to an intermediate position, after which bone cement is introduced into an end region of the first bone bore, whereupon the coupling device is fully introduced into the first bone bore, wherein a bone cement located in the end region is distributed in the femoral head.

20. The method according to claim 19, wherein the bone cement is introduced into the end region of the first bone bore through a cannulated introduction device which introduction device is guided through the nail through an opening through which the adjusting device can be guided through the nail, and is removed from the nail after the introduction of the bone cement, whereupon the adjusting device is inserted into the opening.

21. The method according to claim 16, wherein, distally from a region at which the coupling device can be connected to the nail, a second K-wire is introduced into the femoral neck, in particular at an angle of 0.1° to 15° to the first K-wire, after which a second bone bore is introduced into the femoral neck, wherein the second K-wire serves as a guide, whereupon the second K-wire is removed, after which the adjusting device is introduced into the second bone bore, which adjusting device protrudes past the nail on both sides.
